(19) United States
(12) Patent Application Publication
(10) Pub. No.: US 2026/0359503 A1
(43) Pub. Date: Nov. 12, 2026

(54) ESTIMATION METHOD OF FATIGUE CRACK PROPAGATION RATE OF LAMINATED STRUCTURE

(71) Applicant: Harbin Institute of Technology, Harbin (CN)

(72) Inventors: Jian XIONG, Harbin (CN); Pengcheng XUE, Harbin (CN)

(21) Appl. No.: 19/244,712

(22) Filed: Jun. 20, 2025

(30) Foreign Application Priority Data
Apr. 16, 2025 (CN) 2025104774442

(51) Int. Cl. G06F 30/13

(52) U.S. Cl. CPC G06F 30/13

(57) ABSTRACT

Provided is an estimation method of a fatigue crack propagation rate of a laminated structure which belongs to the field of the fatigue crack prediction technologies of the laminated structures. The present disclosure aims to solve the problem of reliance of the existing prediction of the fatigue crack propagation rate on the support of fatigue test data. The method includes: dividing the laminated structure into an upper sub-beam and a lower sub-beam, calculating a tangential displacement and a normal displacement of the crack interface, calculating a tangential traction force and a normal traction force of the delamination crack interface in combination with an interface fracture toughness of the laminated structure; then, in combination with a resultant force of internal stresses and a resultant force of a bending moment and a shear force of the upper sub-beam and the lower sub-beam, calculating a total strain energy of the laminated structure.

### CROSS-REFERENCE TO RELATED APPLICATION

This application claims priority from the Chinese patent application 2025104774442 filed Apr. 16, 2025, the content of which is incorporated herein in the entirety by reference.

### TECHNICAL FIELD

The present disclosure relates to an estimation method of a fatigue crack propagation rate of a laminated structure and is directed to the field of the fatigue crack prediction technologies of the laminated structures.

### BACKGROUND

In a service process of the engineering materials and structures, the fatigue failure ratio exceeds 80%. Its mechanism research and failure prediction are always the core challenges in the fatigue fracture mechanics. Paris law, as a classical theory, realizes macro-descriptions of the fatigue crack propagation law by constructing a single power law relationship between the fatigue crack propagation rate and the stress intensity factor or energy release rate.

Due to difficulties in carrying out fatigue tests and high costs, the scholars developed numerical methods for simulating the fatigue crack propagation, such as virtual crack closure technique (VCCT) and cohesive elements and the like. Although there are many prediction methods available for fatigue crack propagation, the prediction of the fatigue crack propagation behavior still relies on the support of the fatigue test data. The classical Paris law is a test data-based phenomenological method, which is unable to explain the physical essence of the fatigue crack propagation. It is an empirical formula in which the constants are determined by a huge amount of fatigue test data. Furthermore, although the numerical simulation technology is developed, it is required to obtain basic data by carrying out high-cost and time-consuming fatigue tests to calibrate simulation model parameters, with its prediction accuracy noticeably restricted to the support of the fatigue test data. This technical bottleneck severely restricts the engineering application efficiency of the fatigue life prediction of the laminated structures. Also, the huge workload involved in the fatigue tests leads to prolonged test duration, obviously affecting the structure development process.

### SUMMARY

For the problem of the reliance of the existing prediction of the fatigue crack propagation rate on the support of the fatigue test data, the present disclosure provides an estimation method of a fatigue crack propagation rate of a laminated structure.

The estimation method of the fatigue crack propagation rate of the laminated structure in the present disclosure includes:
dividing the laminated structure into an upper sub-beam and a lower sub-beam based on delamination crack interface, and building a displacement field model of the upper sub-beam and the low sub-beam based on Timoshenko beam theory, and obtaining a formula of a middle surface displacement along a longitudinal direction and a thickness direction and a rotation angle of a middle surface relative to a normal direction; through calculation, obtaining a tangential displacement, a normal displacement of the crack interface and a resultant force of internal stresses and a resultant force of a bending moment and a shear stress of the upper sub-beam and the lower sub-beam;

by using the tangential displacement and the normal displacement of the crack interface in combination with a interface fracture toughness of the laminated structure, calculating a tangential traction force and a normal traction force of the delamination crack interface; then, in combination with the resultant force of the internal stresses and the resultant force of the bending moment and the shear stress of the upper sub-beam and the lower sub-beam, calculating a total strain energy of the laminated structure;

based on the total strain energy of the laminated structure and a potential energy of an external load applied to the laminated structure, establishing an equilibrium equation of the laminated structure, and obtaining a set of governing equations based on the equilibrium equation of the laminated structure;
solving the set of governing equations to obtain theoretically-calculated crack length and cycle number of the laminated structure and applied-load theoretical value of the upper sub-beam and displacement theoretical value of the upper sub-beam under fatigue load of the laminated structure, and obtaining, by calculation, cyclic energy release rate, and further obtaining, by calculation, crack propagation rate.

In the estimation method of the fatigue crack propagation rate of the laminated structure of the present disclosure, the formula of the middle surface displacement along the longitudinal direction and the thickness direction and the rotation angle of the middle surface relative to the normal direction is shown below:

$${u}^{r} ( x , {z}^{r} ) = {u}_{0}^{r} ( x ) - {z}^{r} {φ}^{r} ( x ) \qquad ( 1 )$$

$${w}^{′} ( x , {z}^{r} ) = {w}_{0}^{r} ( x )$$

where u^{r} is a displacement of the upper sub-beam and the lower sub-beam along the longitudinal direction X axis, r=t,b, t is the upper sub-beam, b is the lower sub-beam; w^{r} is a displacement of the upper sub-beam and the lower sub-beam along the thickness direction Z axis; x is the X axis coordinate, z^{r} is the Z axis coordinate of the upper sub-beam and the lower sub-beam,

$${u}_{0}^{r}$$

is the middle surface displacement of the X axis direction of the upper sub-beam and the lower sub-beam,

$${w}_{0}^{r}$$

is the middle surface displacement of the Z axis direction of the upper sub-beam and the lower sub-beam; and φ^{r} is the rotation angle of the middle surface of the upper sub-beam and the lower sub-beam relative to the normal direction.

In the estimation method of the fatigue crack propagation rate of the laminated structure of the present disclosure, the tangential displacement and the normal displacement of the crack interface are calculated as below:

$$Δ {u}_{t} = {u}^{b} ( {z}^{b} = - \frac{{h}_{b}}{2} ) - {u}^{t} ( {z}^{t} = \frac{{h}_{t}}{2} ) \qquad ( 2 )$$

$$Δ {u}_{n} = {w}^{b} ( {z}^{b} = - \frac{{h}_{b}}{2} ) - {w}_{0}^{t} ( {z}^{t} = \frac{{h}_{t}}{2} )$$

where Δuₜ is a tangential displacement of the crack interface, Δuₙ is a normal displacement of the crack interface, h_{b} is a thickness of the lower sub-beam, and hₜ is a thickness of the upper sub-beam.

In the estimation method of the fatigue crack propagation rate of the laminated structure of the present disclosure, the resultant force of the internal stresses and the resultant force of the bending moment and the shear stress of the upper sub-beam and the lower sub-beam are calculated as below:

based on the strain calculation formula of the upper sub-beam and the lower sub-beam:

$${ε}_{x}^{r} ( x , {z}^{r} ) = {u}_{0 , x}^{r} - {z}^{r} {φ}_{, x}^{r} \qquad ( 3 )$$

$${γ}_{x z}^{r} ( x , {z}^{r} ) = {w}_{, x}^{r} - {φ}^{r}$$

where

$${ε}_{x}^{r}$$

is a normal strain of the upper sub-beam and the lower sub-beam,

$${γ}_{x z}^{r}$$

is a shear strain of the upper sub-beam and the lower sub-beam, and in the variable subscript, x is derivation for corresponding variables;
the followings are obtained:

$${N}_{x}^{r} = b {\int }_{- \frac{{h}_{r}}{2}}^{\frac{{h}_{r}}{2}} {σ}_{x}^{r} {dz}^{r} = {A}_{11}^{r} {u}_{0 , x}^{r} - {B}_{11}^{r} {φ}_{, x}^{r} \qquad ( 4 )$$

$${M}_{x}^{r} = b {\int }_{- \frac{{h}_{r}}{2}}^{\frac{{h}_{r}}{2}} {σ}_{x}^{r} {z}^{r} d {z}^{r} = {B}_{11}^{r} {u}_{0 , x}^{r} - {D}_{11}^{r} {φ}_{, x}^{r}$$

$${V}_{xz}^{r} = b {\int }_{- \frac{{h}_{r}}{2}}^{\frac{{h}_{r}}{2}} {τ}_{xz}^{r} d {z}^{r} = {A}_{55}^{r} ( {w}_{, x}^{r} - {ϕ}^{r} )$$

where

$${N}_{x}^{r}$$

is the resultant force of the internal stresses of the upper sub-beam and the lower sub-beam,

$${M}_{x}^{r}$$

is the bending moment of the upper sub-beam and the lower sub-beam, and

$${V}_{x z}^{r}$$

is the resultant force of the shear stresses of the upper sub-beam and the lower sub-beam;
where b is a width of the laminated structure,

$${σ}_{x}^{r}$$

is a normal stress of the X axis direction,

$${τ}_{x z}^{r}$$

is a shear stress in an XZ plane,

$${A}_{11}^{r}$$

is a tensile stiffness of the upper sub-beam and the lower sub-beam,

$${B}_{11}^{r}$$

is a tensile-bending coupling stiffness of the upper sub-beam and the lower sub-beam,

$${D}_{11}^{r}$$

is a bending stiffness of the upper sub-beam and the lower sub-beam, and

$${A}_{55}^{r}$$

is a shear stiffness of the upper sub-beam and the lower sub-beam.

In the estimation method of the fatigue crack propagation rate of the laminated structure of the present disclosure, the interface fracture toughness of the laminated structure is calculated as below:

$${ϕ}_{n} = \frac{3 P W}{2 ( a + Δ ) b} \qquad ( 5 )$$

where φₙ is an interface fracture toughness of the laminated structure, P is an applied load test value of the upper sub-beam, W is a displacement test value of the upper sub-beam, a is a theoretically-calculated crack length of the laminated structure, and Δ is a crack correction length.

In the estimation method of the fatigue crack propagation rate of the laminated structure of the present disclosure, the tangential traction force and the normal traction force of the delamination crack interface are calculated as below:

$${T}_{t} ( D ) = \frac{2 {ϕ}_{n}}{{δ}_{0}} ( 1 - D ) \frac{Δ {u}_{t}}{{δ}_{0}} ( 1 + \frac{Δ {u}_{n}}{{δ}_{0}} ) exp ( - \frac{Δ {u}_{n}}{{δ}_{0}} - \frac{{( Δ {u}_{t} )}^{2}}{{( {δ}_{0} )}^{2}} ) \qquad ( 6 )$$

$${T}_{n} ( D ) = \frac{{ϕ}_{n}}{{δ}_{0}} ( 1 - D ) \frac{Δ {u}_{n}}{{δ}_{0}} exp ( - \frac{Δ {u}_{n}}{{δ}_{0}} - \frac{{( Δ {u}_{t} )}^{2}}{{( {δ}_{0} )}^{2}} )$$

where Tₜ is a tangential traction force, Tₙ is a normal traction force, δ₀ is a characteristic length of a cohesive zone, and D is a damage variable;

D=k∫max(\hat{D}_{c},\hat{D}ₘ)dt (7)

wherein k is a correction coefficient, \hat{D}_{c} is a cycle damage increment, \hat{D}ₘ is a monotonic damage increment, and t is time;

$${\hat{D}}_{c} = \frac{\vert Δ \hat{\overline{u}} \vert}{{δ}_{Σ}} [ \frac{\overline{T}}{{σ}_{m ax}} - \frac{{σ}_{f}}{{σ}_{m ax , 0}} ] H ( Δ \overline{u} - {δ}_{f} ) \qquad ( 8 )$$

$${\hat{D}}_{m} = \frac{\vert Δ \hat{\overline{u}} \vert}{{δ}_{Σ}} H ( Δ \overline{u} - {δ}_{0} ) \qquad ( 9 )$$

wherein Δ\hat{ū} is a cumulative displacement increment, δ_{Σ} is a total displacement amount required for the failure of the cohesive zone, T̅ is a total cohesive traction of the delamination crack interface, σ_{f} is a cohesive fatigue limit, σₘₐₓ is a normal cohesive strength after N load cycles, σ_{max,0} is an interfacial cohesive strength at the time of no damage, H is a Heaviside function, Δū is a cumulative displacement amount, and δ_{f} is a separation displacement corresponding to the cohesive fatigue limit;

$$H ( Δ \overline{u} - {δ}_{0} ) = \{ \begin{matrix}1 , & Δ \overline{u} - {δ}_{0} \geq 0 \\ 0 , & Δ \overline{u} - {δ}_{0} < 0\end{matrix} \qquad ( 10 )$$

wherein the cohesive fatigue limit σ_{f} is determined by a conditional yield stress: defining a parallel line of an elastic response stage of a curve of the normal traction force Tₙ−the normal displacement Δuₙ of the crack interface such that the parallel line is offset as per a designated plastic deformation amount d; the cohesive fatigue limit σ_{f} is jointly determined by dimensionless parameters δ_{f}/δ₀ and σ_{f}/σ_{max,0} at an intersection point of the offset parallel line and the curve of the normal traction force Tₙ−the normal displacement Δuₙ of the crack interface.

In the estimation method of the fatigue crack propagation rate of the laminated structure of the present disclosure, the total strain energy of the laminated structure is calculated as below:

$$U = {\int }_{{V}^{t}} ( {σ}_{x}^{t} {ε}_{x}^{t} + {τ}_{xz}^{t} {γ}_{xz}^{t} ) {dV}^{t} + {\int }_{{V}^{b}} ( {σ}_{x}^{b} {ε}_{x}^{b} + {τ}_{xz}^{b} {γ}_{xz}^{b} ) {dV}^{b} + {\int }_{A} ( {T}_{t} Δ {u}_{t} + {T}_{n} Δ {u}_{n} ) dA \qquad ( 11 )$$

wherein U is the total strain energy of the laminated structure, V^{t} is a volume of the upper sub-beam, V^{b} is a volume of the lower sub-beam, A is an area between crack interface layers;
the potential energy of the external load applied to the laminated structure is expressed as V:

$$V = - {\int }_{0}^{L} {P}^{t} {w}^{t} {δ}_{D} ( x - {x}_{e} ) dx \qquad ( 12 )$$

wherein L is a length of the laminated structure, P^{t} is an applied load theoretical value of the upper sub-beam, δ_{D} is Dirac function, and xₑ is an X axis coordinate of a load application position;

$${δ}_{D} ( x - {x}_{e} ) = \{ \begin{matrix}1 , & x - {x}_{e} = 0 \\ 0 , & x - {x}_{e} \neq 0\end{matrix} . \qquad ( 13 )$$

In the estimation method of the fatigue crack propagation rate of the laminated structure of the present disclosure, based on variational method, the equilibrium equation of the laminated structure is established:

δ(U−V)=0 (14)
wherein δ is a variational symbol;
the following set of governing equations is obtained based on the equilibrium equation of the structure:

$${N}_{x , x}^{t} = - b {T}_{t} \qquad ( 15 )$$

$${V}_{xz , x}^{t} = - b {T}_{n} \qquad ( 16 )$$

$${M}_{x , x}^{t} = {V}_{xz}^{t} - \frac{b {h}_{t}}{2} {T}_{t} \qquad ( 17 )$$

$${N}_{x , x}^{b} = {bT}_{t} \qquad ( 18 )$$

$${V}_{xz , x}^{b} = b {T}_{n} \qquad ( 19 )$$

$${M}_{x , x}^{b} = {V}_{xz}^{b} - \frac{b {h}_{b}}{2} {T}_{t} \qquad ( 20 )$$

$${u}_{0 , x}^{t} = \frac{{D}_{11}^{t} {N}_{x}^{t} - {B}_{11}^{t} {M}_{x}^{t}}{{A}_{11}^{t} {D}_{11}^{t} - {( {B}_{11}^{t} )}^{2}} \qquad ( 21 )$$

$${w}_{0 , x}^{t} = {φ}^{t} + \frac{{V}_{xz}^{t}}{{A}_{55}^{t}} \qquad ( 22 )$$

$${φ}_{, x}^{t} = \frac{{B}_{11}^{t} {N}_{x}^{t} - {A}_{11}^{t} {M}_{x}^{t}}{{A}_{11}^{t} {D}_{11}^{t} - {( {B}_{11}^{t} )}^{2}} \qquad ( 23 )$$

$${u}_{0 , x}^{b} = \frac{{D}_{11}^{b} {N}_{x}^{b} - {B}_{11}^{b} {M}_{x}^{b}}{{A}_{11}^{b} {D}_{11}^{b} - {( {B}_{11}^{b} )}^{2}} \qquad ( 24 )$$

$${w}_{0 , x}^{b} = {φ}^{b} + \frac{{V}_{xz}^{b}}{{A}_{55}^{b}} \qquad ( 25 )$$

$${φ}_{, x}^{b} = \frac{{B}_{11}^{b} {N}_{x}^{b} - {A}_{11}^{b} {M}_{x}^{b}}{{A}_{11}^{b} {D}_{11}^{b} - {( {B}_{11}^{b} )}^{2}} . \qquad ( 26 )$$

In the estimation method of the fatigue crack propagation rate of the laminated structure of the present disclosure, the set of governing equations is solved to obtain the theoretically-calculated crack length a and the cycle number N of the laminated structure and the applied-load theoretical value P^{t} of the upper sub-beam and the displacement theoretical value w^{t} of the upper sub-beam under the fatigue load of the laminated structure;
the cyclic energy release rate is expressed as ΔG:

$$Δ G = \frac{{( {P}_{m ax}^{t} )}^{2} - {( {P}_{m i n}^{t} )}^{2}}{2 b} \frac{dC}{da} \qquad ( 27 )$$

wherein

$${P}_{ma x}^{t}$$

is a maximum value of the applied load theoretical value of the upper sub-beam,

$${P}_{m i n}^{t}$$

is a minimum value of the applied load theoretical value of the upper sub-beam, and C is a compliance of the laminated structure; C=w^{t}/P^{t};
fitting is performed on the theoretically-calculated crack length a and the cycle number N of the laminated structure by using power function to obtain a functional relation and then differential operation is performed on the functional relation to obtain the crack propagation rate da/dN.

In the estimation method of the fatigue crack propagation rate of the laminated structure of the present disclosure, the crack propagation rate da/dN and the cyclic energy release rate ΔG are mapped on a double-logarithmic coordinate system to obtain a curve of the fatigue crack propagation rate of the laminated structure; then based on Paris law, fitting is performed on the curve of the fatigue crack propagation rate to obtain a fitted curve; based on the fitted curve, an actual fatigue crack propagation rate is determined:

$$\frac{da}{dN} = {B ( Δ G )}^{m} \qquad ( 28 )$$

where B is a material-related constant and m is an exponential constant of the Paris law.

The present disclosure has the beneficial effects: in the method of the present disclosure, for the technical problem of high reliance of the prediction of the fatigue crack propagation rate of the laminated structure on the fatigue test data to calibrate the model parameters, without reliance on the fatigue test data, the load displacement response data obtained by simple static tests is used to realize the estimation on the fatigue crack propagation rate of the laminated structure.

The method of the present disclosure solves the problem that the traditional models rely on the fatigue test data. The method has its obvious advantage that without using the fatigue data to calibrate the model parameters, the method can realize effective prediction of the fatigue crack propagation rate by only relying on the strain energy release rate obtained by the quasi-static test. The present disclosure provides an efficient and convenient analysis tool for estimation of the fatigue crack propagation behavior of the laminated structure, significantly improving the calculation efficiency and engineering applicability of the fatigue damage tolerance analysis.

The method of the present disclosure applies to the prediction of the fatigue crack propagation rage of various types of laminated structures, including but not limited to a laminated plate structure of single metal material, and a laminated plate structure of fiber-reinforced composite material; a laminated plate structure of mixture of fiber-reinforced and metal materials; a foam sandwich structure of metal material or composite material, and honeycomb sandwich structure and the like. This method is a general prediction method widely applied to the fatigue failure problem of the laminated structures in the engineering applications.

### BRIEF DESCRIPTIONS OF THE DRAWINGS

FIG. 1 is a schematic diagram illustrating a method of determining a crack correction length Δ in an estimation method of a fatigue crack propagation rate of a laminated structure according to the present disclosure.

FIG. 2 is a schematic diagram of a model of the laminated structure, where x^{t} is an X axis coordinate of an upper sub-beam and x^{b} is an X axis coordinate of a lower sub-beam.

FIG. 3 is a schematic diagram illustrating a cohesive zone model between the upper sub-beam and the lower sub-beam.

FIG. 4 is a schematic diagram illustrating a method of determining a cohesive fatigue limit of the cohesive zone model by a conditional yield stress.

FIG. 5 is a schematic diagram illustrating a constitutive relation of the cohesive zone model in the method of the present disclosure.

FIG. 6 is a schematic diagram of obtaining a function by performing fitting on theoretically-calculated crack length and compliance data using power function, where C=i*a\hat{ }j refers to the form of the fitting equation employed; i and j refer to parameters to be determined by fitting, and i and j in the table are parameter values determined by fitting.

FIG. 7 is a schematic diagram of obtaining a function by performing fitting on cycle number and theoretically-calculated crack length data using power function, where a=I+J*N\hat{ }K; I, J and K are parameters to be determined by fitting and fitted to determine the parameter values.

FIG. 8 is a comparison diagram of the curves of the theoretically-calculated and experimentally-tested fatigue crack propagation rates of the laminated plate structure of composite material.

FIG. 9 is a schematic diagram of geometrical sizes of a honeycomb sandwich structure of composite material of a double-cantilever beam in an embodiment 2.

FIG. 10 is a prediction result diagram of a fatigue crack propagation rate of the honeycomb sandwich structure of composite material of the double-cantilever beam in the embodiment 2.

FIG. 11 is a schematic diagram of geometrical sizes of a foam sandwich structure of a single-cantilever beam in an embodiment 3.

FIG. 12 is a prediction result diagram of a predicted fatigue crack propagation rate of the foam sandwich structure of composite material in the embodiment 3.

FIG. 13 is a schematic diagram of geometric sizes of a laminated plate of composite material of the double-cantilever beam in an embodiment 4.

FIG. 14 is a prediction result diagram of a fatigue crack propagation rate of the laminated plate of composite material of the double-cantilever beam in the embodiment 4.

FIG. 15 is a prediction result diagram of a fatigue crack propagation rate of a laminated plate of interface-toughened composite material of the double-cantilever beam in the embodiment 4

### DETAILED DESCRIPTIONS OF EMBODIMENTS

The technical solutions in the embodiments of the present disclosure will be fully and clearly described below in combination with the drawings in the embodiments of the present disclosure. Apparently, the described embodiments are merely some embodiments of the present disclosure rather than all embodiments. All other embodiments obtained by those skilled in the arts based on these embodiments in the present disclosure without carrying out creative work shall fall within the scope of the present disclosure.

It should be noted that in case of no conflicts, the embodiments and the characteristics in the embodiments in the present disclosure can be mutually combined.

The present disclosure will be further set forth below in combination with the drawings, but the descriptions are not used as limitation to the present disclosure.

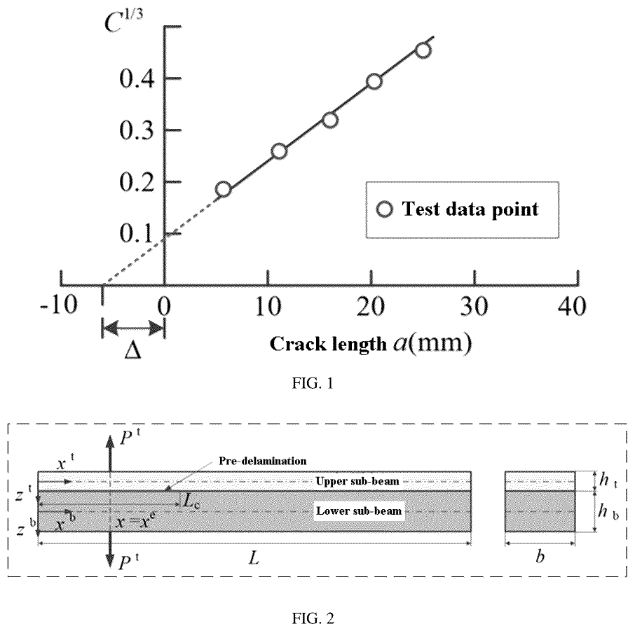

With reference to FIGS. 1 to 8, the present disclosure provides an estimation method of a fatigue crack propagation rate of a laminated structure, which includes:
dividing the laminated structure into an upper sub-beam and a lower sub-beam based on delamination crack interface, and building a displacement field model of the upper sub-beam and the low sub-beam based on Timoshenko beam theory, and obtaining a formula of a middle surface displacement along a longitudinal direction and a thickness direction and a rotation angle of a middle surface relative to a normal direction; through calculation, obtaining a tangential displacement, a normal displacement of the crack interface and a resultant force of internal stresses and a resultant force of a bending moment and a shear stress of the upper sub-beam and the lower sub-beam;
by using the tangential displacement and the normal displacement of the crack interface in combination with a interface fracture toughness of the laminated structure, calculating a tangential traction force and a normal traction force of the delamination crack interface; then, in combination with the resultant force of the internal stresses and the resultant force of the bending moment and the shear stress of the upper sub-beam and the lower sub-beam, calculating a total strain energy of the laminated structure;
based on the total strain energy of the laminated structure and a potential energy of an external load applied to the laminated structure, establishing an equilibrium equation of the laminated structure, and obtaining a set of governing equations based on the equilibrium equation of the laminated structure;
solving the set of governing equations to obtain theoretically-calculated crack length and cycle number of the laminated structure and applied-load theoretical value of the upper sub-beam and displacement theoretical value of the upper sub-beam under fatigue load of the laminated structure, and obtaining, by calculation, cyclic energy release rate, and further obtaining, by calculation, crack propagation rate.

Furthermore, a mechanics model of the laminated structure is built, and an interlayer crack propagation of the laminated structure under monotonic or fatigue load is calculated. The laminated structure is divided based on delamination interface into upper and lower sub-beams which can be respectively made by same or different materials into single layer or multilayer sub-structure, with the model under a local coordinate system as shown in FIG. 2. For the upper and lower sub-beams, the displacement field model is built based on Timoshenko beam theory. The formula of the middle surface displacement along the longitudinal direction and the thickness direction and the rotation angle of the middle surface relative to the normal direction is as follows:

$${u}^{r} ( x , {z}^{r} ) = {u}_{0}^{r} ( x ) - {z}^{r} {φ}^{r} ( x ) \qquad ( 1 )$$

$${w}^{r} ( x , {z}^{r} ) = {w}_{0}^{r} ( x )$$

where u^{r} is a displacement of the upper sub-beam and the lower sub-beam along the longitudinal direction X axis, r=t,b, t is the upper sub-beam, b is the lower sub-beam; w^{r} is a displacement of the upper sub-beam and the lower sub-beam along the thickness direction Z axis; x is the X axis coordinate, z^{r} is the Z axis coordinate of the upper sub-beam and the lower sub-beam,

$${u}_{0}^{r}$$

is the middle surface displacement of the X axis direction of the upper sub-beam and the lower sub-beam,

$${w}_{0}^{r}$$

is the middle surface displacement of the Z axis direction of the upper sub-beam and the lower sub-beam; and φᵣ is the rotation angle of the middle surface of the upper sub-beam and the lower sub-beam relative to the normal direction.

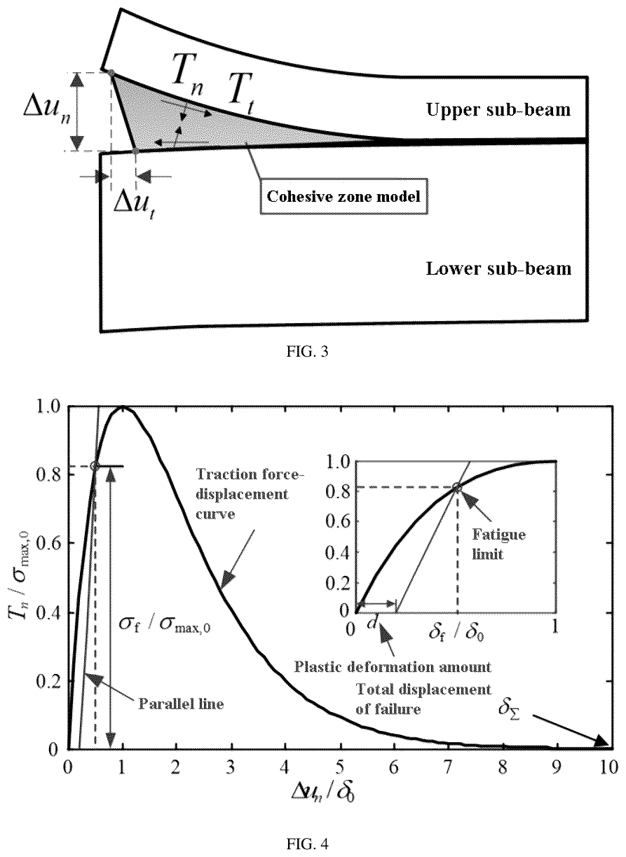

The delamination interface of the laminated structure represents the constitutive relation between upper and lower surfaces by using the cohesive zone model, as shown in FIG. 3. The tangential displacement and the normal displacement of the crack interface are calculated as below:

$$Δ {u}_{t} = {u}^{b} ( {z}^{b} = - \frac{{h}_{b}}{2} ) - {u}^{t} ( {z}^{t} = \frac{{h}_{t}}{2} ) \qquad ( 2 )$$

$$Δ {u}_{n} = {w}^{b} ( {z}^{b} = - \frac{{h}_{b}}{2} ) - {w}_{0}^{t} ( {z}^{t} = \frac{{h}_{t}}{2} )$$

where Δuₜ is the tangential displacement of the crack interface, Δuₙ is the normal displacement of the crack interface, h_{b} is the thickness of the lower sub-beam, and hₜ is the thickness of the upper sub-beam.

Furthermore, the resultant force of the internal stresses and the resultant force of the bending moment and the shear stress of the upper sub-beam and the lower sub-beam are calculated as below:
based on the strain calculation formula of the upper sub-beam and the lower sub-beam:

$${ε}_{x}^{r} ( x , {z}^{r} ) = {u}_{0 , x}^{r} - {z}^{r} {φ}_{, x}^{r} \qquad ( 3 )$$

$${γ}_{x z}^{r} ( x , {z}^{r} ) = {w}_{x}^{r} - {φ}^{r}$$

where

$${ε}_{x}^{r}$$

is a normal strain of the upper sub-beam and the lower sub-beam,

$${γ}_{x z}^{r}$$

is a shear strain of the upper sub-beam and the lower sub-beam, and in the variable subscript, x is derivation for corresponding variables;
the followings are obtained:

$${N}_{x}^{r} = b {\int }_{- \frac{{h}_{r}}{2}}^{\frac{{h}_{r}}{2}} {σ}_{x}^{r} {dz}^{r} = {A}_{11}^{r} {u}_{0 , x}^{r} - {B}_{11}^{r} {ϕ}_{, x}^{r} \qquad ( 4 )$$

$${M}^{r} = b {\int }_{- \frac{{h}_{r}}{2}}^{\frac{{h}_{r}}{2}} {σ}_{x}^{r} {z}^{r} d {z}^{r} = {B}_{11}^{r} {u}_{0 , x}^{r} - {D}_{11}^{r} {φ}_{, x}^{2}$$

$${V}_{xz}^{r} = b {\int }_{- \frac{{h}_{r}}{2}}^{\frac{{h}_{r}}{2}} {τ}_{xz}^{r} d {z}^{r} = {A}_{55}^{r} ( {w}_{, x}^{r} - {φ}^{r} )$$

where

$${N}_{x}^{r}$$

is the resultant force of the internal stresses of the upper sub-beam and the lower sub-beam,

$${M}_{x}^{r}$$

is the bending moment of the upper sub-beam and the lower sub-beam, and

$${V}_{xz}^{r}$$

is the resultant force of the shear stresses of the upper sub-beam and the lower sub-beam;
where b is a width of the laminated structure,

$${σ}_{x}^{r}$$

is a normal stress of the X axis direction,

$${τ}_{x z}^{r}$$

is a shear stress in an XZ plane,

$${A}_{11}^{r}$$

is a tensile stiffness of the upper sub-beam and the lower sub-beam,

$${B}_{11}^{r}$$

is a tensile-bending coupling stiffness of the upper sub-beam and the lower sub-beam,

$${D}_{11}^{r}$$

is a bending stiffness of the upper sub-beam and the lower sub-beam, and

$${A}_{55}^{r}$$

is a shear stiffness of the upper sub-beam and the lower sub-beam.

Double-cantilever beam tensile test under monotonic load is performed on the laminated structure with the prefabricated delamination crack to obtain the load displacement response data of the structure, and based on modified beam theory, the interface fracture toughness of the laminated structure is calculated. The interface fracture toughness of the laminated structure is calculated as below:

$${ϕ}_{n} = \frac{3 P W}{2 ( a + Δ ) b} \qquad ( 5 )$$

where φₙ is an interface fracture toughness of the laminated structure, P is an applied load test value of the upper sub-beam, W is a displacement test value of the upper sub-beam, a is a theoretically-calculated crack length of the laminated structure, and Δ is a crack correction length.

In combination with FIG. 1, the crack correction length Δ is obtained by extrapolating the linear fitting equation of the compliance cube root C^{1/3} and the theoretically-calculated crack length a to C^{1/3}=0.

The tangential traction force and the normal traction force of the delamination crack interface are calculated as below:

$${T}_{t} ( D ) = \frac{2 {ϕ}_{n}}{{δ}_{0}} ( 1 - D ) \frac{Δ {u}_{t}}{{δ}_{0}} ( 1 + \frac{Δ {u}_{n}}{{δ}_{0}} ) exp ( - \frac{Δ {u}_{n}}{{δ}_{0}} - \frac{{( Δ {u}_{t} )}^{2}}{{( {δ}_{0} )}^{2}} ) \qquad ( 6 )$$

$${T}_{n} ( D ) = \frac{{ϕ}_{n}}{{δ}_{0}} ( 1 - D ) \frac{Δ {u}_{n}}{{δ}_{0}} exp ( - \frac{Δ {u}_{n}}{{δ}_{0}} - \frac{{( Δ {u}_{t} )}^{2}}{{( {δ}_{0} )}^{2}} )$$

where Tₜ is the tangential traction force, Tₙ is the normal traction force, φₙ equal to a fracture toughness obtained in the static test; δ₀ is a characteristic length of a cohesive zone, and D is a damage variable, representing a degradation degree of the interface performance.

Under the fatigue load, the damage variable D is expressed as below:

D=k∫max(\hat{D}_{c},\hat{D}ₘ)dt (7)
where k is a correction coefficient valued as 0.01, \hat{D}_{c} is a cycle damage increment, \hat{D}ₘ is a monotonic damage increment, and t is time;

$${\hat{D}}_{c} = \frac{\vert Δ \hat{\overline{u}} \vert}{{δ}_{\sum }} [ \frac{\overline{T}}{{σ}_{max}} - \frac{{σ}_{f}}{{σ}_{max , 0}} ] H ( Δ \overline{u} - {δ}_{f} ) \qquad ( 8 )$$

$${\hat{D}}_{m} = \frac{\vert Δ \hat{\overline{u}} \vert}{{δ}_{\sum }} H ( Δ \hat{\overline{u}} - {δ}_{0} ) \qquad ( 9 )$$

where Δ\hat{ū} is a cumulative displacement increment, δ_{Σ} is a total displacement amount required for the failure of the cohesive zone, T̅ is a total cohesive traction of the delamination crack interface

$$\overline{T} = \sqrt{{T}_{n}^{2} + {T}_{t}^{2} / ( 2 e )} ;$$

σₜ is a cohesive fatigue limit, σₘₐₓ is a normal cohesive strength after N load cycles, σ_{max,0} is an interfacial cohesive strength at the time of no damage, σₘₐₓ=σ_{max,0}(1−D); H is a Heaviside function; only when the value in the parentheses is greater than or equal to 0, the function value is 1, and otherwise, the function value is 0; Δū is a cumulative displacement amount, and δₜ is a separation displacement corresponding to the cohesive fatigue limit;

$$H ( Δ \overline{u} - {δ}_{0} ) = \{ \begin{matrix}1 , & Δ \overline{u} - {δ}_{0} \geq 0 \\ 0 , & Δ \overline{u} - {δ}_{0} < 0\end{matrix} . \qquad ( 10 )$$

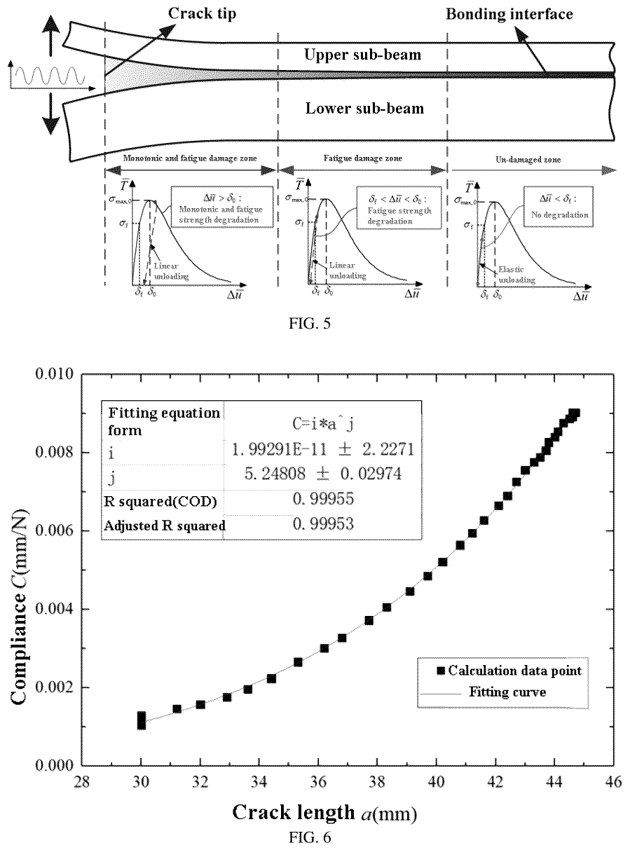

Furthermore, the cohesive fatigue limit of the cohesive zone model is defined by a conditional yield stress: specifically, defining a parallel line of an elastic response stage of a curve of the normal traction force Tₙ−the normal displacement Δuₙ of the crack interface such that the parallel line is offset as per a designated plastic deformation amount d; the cohesive fatigue limit σ_{f} is jointly determined by two dimensionless parameters (δ_{f}/δ₀ and σ_{f}/σ_{max,0}) at an intersection point of the offset parallel line and the traction force-displacement curve, as shown in FIG. 4. The total displacement amount δ_{Σ} of the failure of the cohesive zone is defined as 10δ₀. The traction force value corresponding to the displacement point degrades to 0.1% of the initial cohesive strength. The schematic diagram of the finally-determined constitutive relation of the cohesive zone model is as shown in FIG. 5. When Δū>δ₀, monotonic damage and fatigue damage are generated at the same time in the cohesive zone. The increments of the two damages are present simultaneously, and the greater of them is the final damage increment of the current cycle, and the unloading path returns from the linear unloading of the current state point to the traction force zero point. When δₜ<Δū<δ₀, the cohesive traction force is more than the cohesive fatigue limit but less than the cohesive strength. The cohesive zone only generates fatigue damage, and the unloading path returns from the linear unloading of the current state point to the traction zero point. When Δū<δ_{f}, the cohesive traction force is in the elastic section and does not generate damage, and the unloading path returns to the origin along the elastic section.

The total strain energy of the laminated structure is calculated as below:

$$U = {\int }_{{V}^{t}} ( {σ}_{x}^{t} {ε}_{x}^{t} , + {τ}_{x 𝓏}^{t} {γ}_{x 𝓏}^{t} ) {dV}^{t} + {\int }_{{V}^{b}} ( {σ}_{x}^{b} {ε}_{x}^{b} , + {τ}_{x 𝓏}^{b} {γ}_{x 𝓏}^{b} ) {dV}^{b} + {\int }_{A} ( {T}_{t} Δ {u}_{t} + {T}_{n} Δ {u}_{n} ) dA \qquad ( 11 )$$

where U is the total strain energy of the laminated structure, V^{t} is a volume of the upper sub-beam, V^{b} is a volume of the lower sub-beam, A is an area between crack interface layers;
the potential energy of the external load applied to the laminated structure is expressed as V:

$$V = - {\int }_{0}^{L} {P}^{t} {w}^{t} {δ}_{D} ( x - {x}_{e} ) dx \qquad ( 12 )$$

where L is a length of the laminated structure, P^{t} is an applied load theoretical value of the upper sub-beam, β_{D} is Dirac function; only when the value in parentheses is 0, the function value is 1 and otherwise, the function value is 0; and x_{c} is an X axis coordinate of a load application position;

$${δ}_{D} ( x - {x}_{e} ) = \{ \begin{matrix}1 , & x - {x}_{e} = 0 \\ 0 , & x - {x}_{e} \neq 0\end{matrix} . \qquad ( 13 )$$

Based on the minimum potential energy principle, by variational method, the equilibrium equation of the laminated structure is established:

δ(U−V)=0 (14)
where δ is a variational symbol;
the following set of governing equations is obtained based on the equilibrium equation of the structure:

$${N}_{x , x}^{t} = - b {T}_{t} \qquad ( 15 )$$

$${V}_{x𝓏 , x}^{t} = - b {T}_{n} \qquad ( 16 )$$

$${M}_{x , x}^{t} = {V}_{x𝓏}^{t} - \frac{b {h}_{t}}{2} {T}_{t} \qquad ( 17 )$$

$${N}_{x , x}^{b} = {bT}_{t} \qquad ( 18 )$$

$${V}_{x𝓏 , x}^{b} = b {T}_{n} \qquad ( 19 )$$

$${M}_{x , x}^{b} = {V}_{x𝓏}^{b} - \frac{b {h}_{b}}{2} {T}_{t} \qquad ( 20 )$$

$${u}_{0 , x}^{t} = \frac{{D}_{11}^{t} {N}_{x}^{t} - {B}_{11}^{t} {M}_{x}^{t}}{{A}_{11}^{t} {D}_{11}^{t} - {( {B}_{11}^{t} )}^{2}} \qquad ( 21 )$$

$${w}_{0 , x}^{t} = {φ}^{t} + \frac{{V}_{x𝓏}^{t}}{{A}_{55}^{t}} \qquad ( 22 )$$

$${φ}_{, x}^{t} = \frac{{B}_{11}^{t} {N}_{x}^{t} - {A}_{11}^{t} {M}_{x}^{t}}{{A}_{11}^{t} {D}_{11}^{t} - {( {B}_{11}^{t} )}^{2}} \qquad ( 23 )$$

$${u}_{0 , x}^{b} = \frac{{D}_{11}^{b} {N}_{x}^{b} - {B}_{11}^{b} {M}_{x}^{b}}{{A}_{11}^{b} {D}_{11}^{b} - {( {B}_{11}^{b} )}^{2}} \qquad ( 24 )$$

$${w}_{0 , x}^{b} = {φ}^{b} + \frac{{V}_{x𝓏}^{b}}{{A}_{55}^{b}} \qquad ( 25 )$$

$${φ}_{, x}^{b} = \frac{{B}_{11}^{b} {N}_{x}^{b} - {A}_{11}^{b} {M}_{x}^{b}}{{A}_{11}^{b} {D}_{11}^{b} - {( {B}_{11}^{b} )}^{2}} . \qquad ( 26 )$$

By numerical calculation software, the above first-order ordinary differential governing equation set is solved to obtain the theoretically-calculated crack length a and the cycle number N of the laminated structure and the applied-load theoretical value P^{t} of the upper sub-beam and the displacement theoretical value w^{t} of the upper sub-beam under the fatigue load of the laminated structure;
by using the load and displacement obtained by theoretical method, the cyclic energy release rate is calculated, which is expressed as ΔG:

$$Δ G = \frac{{( {P}_{max}^{t} )}^{2} - {( {P}_{min}^{t} )}^{2}}{2 b} \frac{dC}{da} \qquad ( 27 )$$

where

$${P}_{max}^{t}$$

is a maximum value of the applied load theoretical value of the upper sub-beam,

$${P}_{min}^{t}$$

is a minimum value of the applied load theoretical value of the upper sub-beam, and C is a compliance of the laminated structure; C=w^{t}/P^{t};

$$\frac{dC}{da}$$

is obtained by performing differential operation on a functional relation obtained by performing fitting on the theoretically-calculated crack length and compliance data.

Fitting is performed on the theoretically-calculated crack length a and the cycle number N of the laminated structure by using power function to obtain a functional relation and then differential operation is performed on the functional relation to obtain the crack propagation rate da/dN.

Furthermore, the crack propagation rate da/dN and the cyclic energy release rate ΔG are mapped on a double-logarithmic coordinate system to obtain the curve of the fatigue crack propagation rate of the laminated structure; then based on Paris law, fitting is performed on the curve of the fatigue crack propagation rate to obtain a fitted curve; based on the fitted curve, an actual fatigue crack propagation rate is determined:

$$\frac{d a}{d N} = {B ( Δ G )}^{m} \qquad ( 28 )$$

where B is a material-related constant and m is an exponential constant of the Paris law.

This method of the present disclosure is applicable to the prediction of the fatigue crack propagation rate of several types of laminated structures. The laminated structure and sandwich structure of fiber-reinforced composite material are structures most widely used in various fields at present. The honeycomb sandwich structures of composite material are mostly used for the parts such as cowling, aileron and rudder of the passenger planes; the foam sandwich structures also have mature applications in ships and rail transits. Against the background of energy saving, emission reduction and energy transformation, the composite material has become the first choice for the materials of wind turbine blades. Next, the fatigue crack propagation rate is calculated by the method of the present disclosure for several typical laminated structures to show the applicability of the method of the present disclosure in the fatigue performance development of the engineering structures as well as the application value of this method in prediction of the fatigue crack propagation rate of the laminated structures.

The calculation process of the method of the present disclosure can be implemented on a general computer and the calculated data can be processed to obtain the prediction result of the fatigue crack propagation rate of the structures. The fatigue crack propagation rate of the following structures is calculated on the computers with the following configurations: Intel Core processor, processor main frequency 3.2 G; memory capacity 8 G; hard drive capacity 500 G.

### Embodiment 1

At step S1, a laminated plate structure of composite material with the prefabricated delamination crack is prepared as shown in FIG. 2. The test specimen has a total length of L=200 mm, a width of b=25 mm and a total thickness of 4.5 mm. The prefabricated delamination crack of the upper and lower sub-beams is equally divided into sub-beams with same thickness, and the length of the prefabricated crack is Lc=30 mm. The basic mechanic properties of the composite material used are shown in Table 1. A tensile load is applied to the laminated plate structure of composite material with prefabricated delamination crack at the position of x_{c}=25 mm to perform double-cantilever beam tensile test under monotonic load so as to obtain the load displacement response data of the structure. The interface fracture toughness of the laminated plate structure of composite material is calculated in the formula (5).

TABLE 1
Basic mechanic properties of the
laminated plate of composite material

| Parallel | Vertical | In-plane | |
|---|---|---|---|
| fiber direction | fiber direction | shear | Poisson's |
| Elastic modulus | Elastic modulus | modulus | Ratio |
| E₁ (GPa) | E₂ (GPa) | G₁₂ (GPa) | ν₁₂ |
| 57.8 | 57.8 | 19.9 | 0.3 |

At step S2, based on the geometric parameters and material parameters of the structure in the step S1, a mechanic model of the laminated plate structure of composite material is built; and the basic mechanic properties of the laminated plate of composite material are input into the formula (4) and the interface fracture toughness obtained by the quasi-static tensile test is input into the formula (6).

At step S3, the cohesive fatigue limit of the cohesive zone model is defined by a conditional yield stress. As shown in FIG. 3, a parallel line of the elastic response stage of the traction force-displacement curve is drawn such that the parallel line is offset as per a designated plastic deformation amount d; the value of the conditional yield stress is jointly determined by two dimensionless parameters (δ_{f}/δ₀ and σ_{f}/σ_{max,0}) at the intersection point of the offset line and the traction force-displacement curve, and the values of the two dimensionless parameters are calculated based on different plastic deformation amounts d, as shown in Table 2.

TABLE 2
Values of the dimensionless parameters corresponding to different plastic
deformation amounts

| Plastic | | | | | | | | | |
|---|---|---|---|---|---|---|---|---|---|
| deformation | | | | | | | | | |
| amount d | 0 | 1% | 5% | 10% | 20% | 30% | 40% | 50% | 60% |
| δ_{f}/δ₀ | 0 | 0.1026 | 0.2370 | 0.3438 | 0.5047 | 0.6369 | 0.7548 | 0.8642 | 0.9677 |
| σ_{f}/σ_{max,0} | 0 | 0.2516 | 0.5083 | 0.6626 | 0.8282 | 0.9157 | 0.9646 | 0.9899 | 0.9995 |

At step S4, the formulas (15)-(26) of the set of governing equations are solved to obtain the theoretically-calculated interlayer crack length a, the cycle number N and the applied-load theoretical value P^{t} of the upper sub-beam and the displacement theoretical value w^{t} of the upper sub-beam under the fatigue load of the laminated plate structure of composite material.

At step S5, by using the load and displacement data obtained in the step S4, the cyclic energy release rate ΔG of the structure under the fatigue load is calculated in the formula (27), where the dC/da in the formula is obtained by performing differential operation on a functional relation obtained by performing fitting on the theoretically-calculated crack length and compliance data, as shown in FIG. 6.

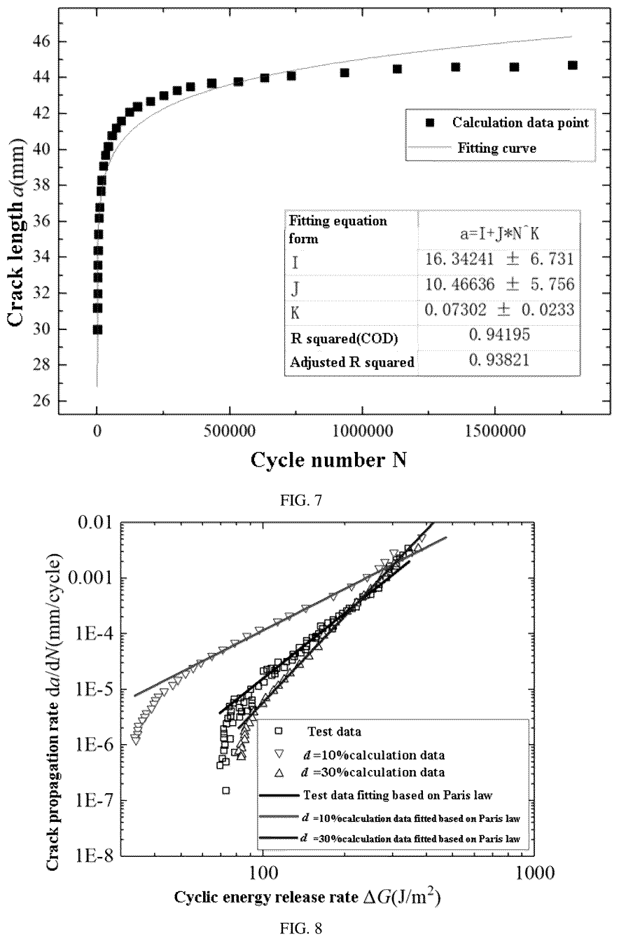

At step S6, by using the crack propagation length and the cycle number data obtained in the step S4, fitting is performed on the calculation data by power function to obtain a functional relation, and then differential operation is performed on the functional relation to obtain the crack propagation rate da/dN as shown in FIG. 7.

At step S7, the theoretically-calculated crack propagation rate da/dN and cyclic energy release rate ΔG are mapped on a double-logarithmic coordinate system to obtain the curve of the fatigue crack propagation rate of the laminated plate structure of composite material, as shown in FIG. 8. Based on Paris law, fitting is performed on the curves of the theoretically-calculated and experimentally-tested fatigue crack propagation rates to obtain an exponential constant of the Paris law, as shown in Table 3.

TABLE 3
The exponential constant m of the Paris Law by
theoretical calculation and experimental test

| | Theoretical | Theoretical |
|---|---|---|
| Experimental | calculation | calculation |
| test | d = 10% | d = 30% |
| 3.9 | 2.5 | 5.2 |

### Embodiment 2

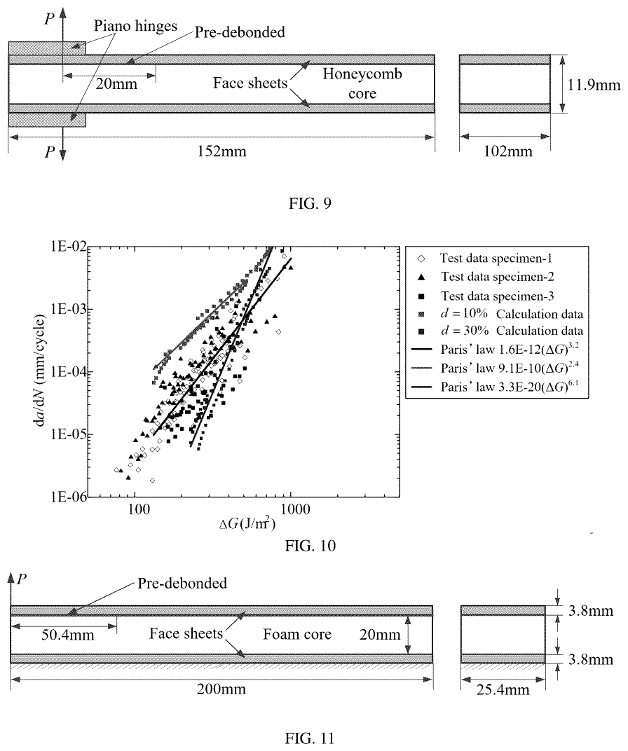

The method of the present disclosure is used for the honeycomb sandwich structure of composite material to predict the fatigue crack propagation rate. The honeycomb sandwich structure of composite material is made into the structure of double-cantilever beam, and a debonding region is pre-prepared between upper panel and core. A pre-debonding length between a loading position of a load and a crack tip is 20 mm, and the specific sizes of the honeycomb sandwich structure of the double-cantilever beam are as shown in FIG. 9. Firstly, quasi-static tensile test is carried out on the honeycomb sandwich structure of the double-cantilever beam to obtain a load displacement response data of the structure, and based on modified beam theory, the interface fracture toughness of the honeycomb sandwich structure is calculated as 1180 J/m². Then, fatigue test is carried out on the honeycomb sandwich structure of the double-cantilever beam, with the fatigue test frequency being 4 Hz and the load ratio of the applied load being 0.1. The fatigue load test data is used as a final verification approach to test the effectiveness and applicability of the method of the present disclosure.

The main flow for calculation of the fatigue crack propagation rate of the honeycomb sandwich structure of the double-cantilever beam by the method of the present disclosure includes: based on Timoshenko beam theory, performing modeling on the honeycomb sandwich structure; inputting the interface fracture toughness of the honeycomb sandwich structure obtained by quasi-static tensile test into the established cohesive zone model, and using the cohesive zone model to represent the mechanical behaviors of the bonding interface of the upper and lower panels and the core; based on variational method, deriving the equilibrium equation of the honeycomb sandwich structure so as to obtain a set of governing equations of the honeycomb sandwich structure; performing numerical solving on the set of governing equations to obtain the theoretically-calculated crack length and cycle number as well as the load theoretical value and displacement theoretical value applied to the upper sub-beam; finally, processing data to obtain the fatigue crack propagation rate of the structure and performing fitting on the data based on Paris law.

The fatigue crack propagation rate of the honeycomb sandwich structure of composite material predicted by the method of the present disclosure is as shown in FIG. 10. FIG. 10 shows comparison of the prediction result and the fatigue crack propagation rate obtained by real test. From the results of FIG. 10, it can be seen that, without performing calibration on the model parameters using the fatigue test data, the method of the present disclosure successfully predicts the distribution range of the fatigue crack propagation rate of the honeycomb sandwich structure of composite material, and the fatigue crack propagation rate obtained by real test is within the predicted range. It is proved that the method of the present disclosure can, without performing calibration on the model parameters using the fatigue test data, predict the distribution range of the fatigue crack propagation rate of the honeycomb sandwich structure of composite material by only relying on the quasi-static test result, which solves the fundamental problem of predicting the fatigue crack propagation rate by using the pre-obtained fatigue test data in the prior arts.

### Embodiment 3

The method of the present disclosure is applied to the prediction of the fatigue crack propagation rate of the foam sandwich structure of composite material. The foam sandwich structure of composite material is made into the structure of single-cantilever beam and a debonding region is pre-prepared between upper panel and core. A pre-debonding length between a loading position of a load and a crack tip is 50.4 mm, and the specific sizes of the foam sandwich structure of the single-cantilever beam are as shown in FIG. 11. Firstly, quasi-static tensile test is carried out on the foam sandwich structure of the single-cantilever beam to obtain a load displacement response data of the structure, and based on modified beam theory, the interface fracture toughness of the foam sandwich structure is calculated as 722 J/m². Then, fatigue test is carried out on the foam sandwich structure of the single-cantilever beam, with the fatigue test frequency being 2 Hz and the load ratio of the applied load being 0.1. The fatigue load test data is used as a final verification approach to test the effectiveness and applicability of the method of the present disclosure.

The main calculation flow is the same as in the embodiment 2 and no redundant descriptions are made herein.

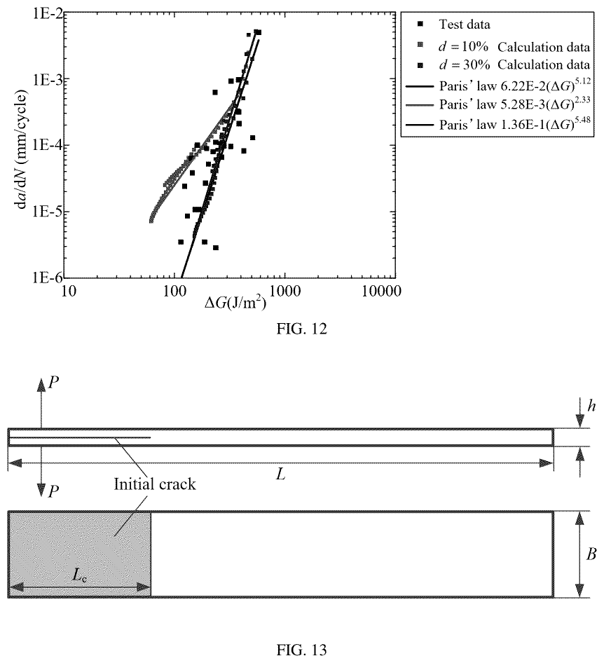

The fatigue crack propagation rate of the foam sandwich structure of composite material predicted by the method of the present disclosure is as shown in FIG. 12. FIG. 12 shows comparison of the prediction result and the fatigue crack propagation rate obtained by real test. From the results of FIG. 12, it can be seen that, without performing calibration on the model parameters using the fatigue test data, the method of the present disclosure also successfully predicts the distribution range of the fatigue crack propagation rate of the foam sandwich structure of composite material, and the fatigue crack propagation rate obtained by real test is within the predicted range.

### Embodiment 4

The method of the present disclosure is applied to prediction of the fatigue crack propagation rate of the laminated plate structure of carbon fiber composite material. The material for preparing the laminated plate structure of carbon fiber composite material is unidirectional carbon fiber/epoxy composite material of UT500/111, and is made into the structure of double-cantilever beam. Two types of laminated plates are prepared respectively: one is a laminated plate of ordinary composite material without interlayer toughening, and the other is a laminated plate of composite material with interlayer toughening carried out using thermoplastic ionomer; a delamination region is pre-prepared at a middle thickness of the two laminated plates of composite material, with the length L=140 mm, width B=20 mm, thickness h=3 mm and initial crack length L_{c}=20 mm as shown in FIG. 13. Firstly, quasi-static tensile test is carried out on the laminated plate of composite material of the double-cantilever beam to obtain a load displacement response data of the structure, and based on modified beam theory, the interface fracture toughnesses of the two laminated plates of composite material are calculated respectively as 150 J/m² and 490 J/m². Then, fatigue test is carried out on the laminated plates of composite material of the double-cantilever beam, with the fatigue test frequency being 10 Hz and the load ratio of the applied load being 0.2. The fatigue load test data is used as a final verification approach to test the effectiveness and applicability of the method of the present disclosure. In other words, the method is compared with fatigue test carried out on the laminated structure, with the fatigue test frequency being 4 Hz and the load ratio of an applied load being 0.1, wherein fatigue load test data is used as a verification approach to test the effectiveness and applicability of the method of the present disclosure, and the method is applied to a prediction of a fatigue crack propagation rate of the laminated structures in aerospace vehicles under fatigue load, and provide an analysis tool for the development, designing, optimization and damage tolerance evaluation of the structures in aerospace fields.

The main calculation flow is the same as in the embodiment 2 and no redundant descriptions are made herein.

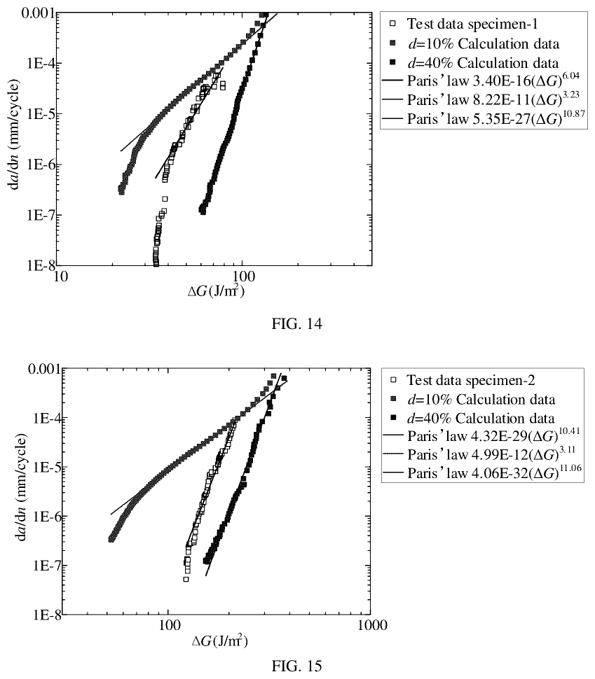

The fatigue crack propagation rates of the two laminated plates of composite material predicted by the method of the present disclosure are as shown in FIGS. 14 and 15. FIGS. 14 and 15 show comparison of the prediction result and the fatigue crack propagation rate obtained by real test. From the results of FIGS. 14 and 15, it can be seen that, without performing calibration on the model parameters using the fatigue test data, the method of the present disclosure also successfully predicts the distribution range of the fatigue crack propagation rate of the laminated plates of composite material, and it is within the range of the fatigue crack propagation rate obtained by real test.

In summary, it can be seen that the method of the present disclosure can, without performing calibration on the model parameters using the fatigue test data, predict the distribution range of the fatigue crack propagation rate of multiple laminated structures by only relying on the quasi-static test result, which solves the fundamental problem of predicting the fatigue crack propagation rate by using the pre-obtained fatigue test data in the prior arts, shows the applicability of the present disclosure in the fatigue performance development of the engineering structures, and proves the application value of this method in prediction of the fatigue crack propagation rate of the laminated structures.

The application fields of the present disclosure will be exemplified below.
1) Aerospace field: the method of the present disclosure is applied to the prediction of the fatigue crack propagation rate of the laminated plate and sandwich plate structures of composite material in the aerospace vehicles under fatigue load, and can provide an efficient and convenient analysis tool for the development, designing, optimization and damage tolerance evaluation of the structures in the aerospace fields.
2) Transportation field: due to its lightweight and corrosion resistance characteristics, the structures of composite material are widely applied in the ship structure building. The foam sandwich plates are often used for the high-speed trains to reduce the train weight and increase its running speed. The method of the present disclosure can be used to predict the fatigue crack propagation rate of the laminated plate and sandwich structures in the main bearing structures and secondary bearing structures of the ships and speed trains, providing guidance in structure security, optimized designing, and maintenance and promoting the application development of the composite materials in the transportation field.
3) Wind power field: due to the lightweight and unique airfoil designing requirements of the wind turbine blades, the composite materials have unique advantages in this field. The method of the present disclosure can be used for the prediction of the fatigue crack propagation rate of the structures of composite material with delamination damage in the wind turbine blades, providing a theoretical prediction approach for the life evaluation and damage tolerance designing of the wind turbine blades and reducing the maintenance costs and improving the economic benefits.
4) Architectural structure field: In the modern civil engineering structures, a fiber-reinforced composite material is usually bonded on the timber and reinforced concrete structures for reinforcement. The method of the present disclosure can be used for the prediction of the fatigue crack propagation rate of the bonding interface of the base beam and the reinforcing beam of composite material, providing basis for optimization of reinforcement plans and extending the service life of the structures.

Although the present disclosure is described by referring to the specific embodiments herein, it should be understood that these embodiments are merely examples of the principles and applications of the present disclosure. Therefore, it should be understood that many modifications can be made to the illustrative embodiments and other arrangements can be designed as long as the spirit and scope of the present disclosure defined in the appended claims are observed. Furthermore, it should be understood that different dependent claims and the characteristics in the specification can be combined in a manner different from the original claims. It can also be understood that the characteristics described in combination with individual embodiments can be used in other embodiments.

1. An estimation method of a fatigue crack propagation rate of a laminated structure, comprising:
dividing the laminated structure into an upper sub-beam and a lower sub-beam based on delamination crack interface, and building a displacement field model of the upper sub-beam and the low sub-beam based on Timoshenko beam theory, and obtaining a formula of a middle surface displacement along a longitudinal direction and a thickness direction and a rotation angle of a middle surface relative to a normal direction; through calculation, obtaining a tangential displacement, a normal displacement of the crack interface and a resultant force of internal stresses and a resultant force of a bending moment and a shear stress of the upper sub-beam and the lower sub-beam;
by using the tangential displacement and the normal displacement of the crack interface in combination with a interface fracture toughness of the laminated structure, calculating a tangential traction force and a normal traction force of the delamination crack interface; then, in combination with the resultant force of the internal stresses and the resultant force of the bending moment and the shear stress of the upper sub-beam and the lower sub-beam, calculating a total strain energy of the laminated structure;
based on the total strain energy of the laminated structure and a potential energy of an external load applied to the laminated structure, establishing a equilibrium equation of the laminated structure, and obtaining a set of governing equations based on the equilibrium equation of the laminated structure;
solving the set of governing equations to obtain theoretically-calculated crack length and cycle number of the laminated structure and applied-load theoretical value of the upper sub-beam and displacement theoretical value of the upper sub-beam under fatigue load of the laminated structure, and obtaining, by calculation, cyclic energy release rate, and further obtaining, by calculation, crack propagation rate, comparing the method with fatigue test carried out on the laminated structure, with the fatigue test frequency being 4 Hz and the load ratio of an applied load being 0.1, wherein fatigue load test data is used as a verification approach to test the effectiveness and applicability of the method of the present disclosure, and the method is applied to a prediction of a fatigue crack propagation rate of the laminated structures in aerospace vehicles under fatigue load, and provide an analysis tool for the development, designing, optimization and damage tolerance evaluation of the structures in aerospace fields.

2. The estimation method of the fatigue crack propagation rate of the laminated structure according to claim 1, wherein,
the formula of the middle surface displacement along the longitudinal direction and the thickness direction and the rotation angle of the middle surface relative to the normal direction is shown below:
$${u}^{r} ( x , {𝓏}^{r} ) = {u}_{0}^{r} ( x ) - {𝓏}^{r} {φ}^{r} ( x ) {w}^{r} ( x , {𝓏}^{r} ) = {w}_{0}^{r} ( x ) \qquad ( 1 )$$
wherein u^{r} is a displacement of the upper sub-beam and the lower sub-beam along the longitudinal direction X axis, r=t,b, t is the upper sub-beam, b is the lower sub-beam; w^{r} is a displacement of the upper sub-beam and the lower sub-beam along the thickness direction Z axis; x is the X axis coordinate, z^{r} is the Z axis coordinate of the upper sub-beam and the lower sub-beam,
$${u}_{0}^{r}$$
is the middle surface displacement of the X axis direction of the upper sub-beam and the lower sub-beam,
$${w}_{0}^{r}$$
is the middle surface displacement of the Z axis direction of the upper sub-beam and the lower sub-beam; and φ^{r} is the rotation angle of the middle surface of the upper sub-beam and the lower sub-beam relative to the normal direction.

3. The estimation method of the fatigue crack propagation rate of the laminated structure according to claim 2, wherein, the tangential displacement and the normal displacement of the crack interface are calculated as below:
$$Δ {u}_{t} = {u}^{b} ( {𝓏}^{b} = - \frac{{h}_{b}}{2} ) - {u}^{t} ( {𝓏}^{t} = \frac{{h}_{t}}{2} ) Δ {u}_{n} = {w}^{b} ( {𝓏}^{b} = - \frac{{h}_{b}}{2} ) - {w}_{0}^{t} ( {𝓏}^{t} = \frac{{h}_{t}}{2} ) \qquad ( 2 )$$
wherein Δuₜ is a tangential displacement of the crack interface, Δuₙ is a normal displacement of the crack interface, h_{b}, is a thickness of the lower sub-beam, and hₜ is a thickness of the upper sub-beam.

4. The estimation method of the fatigue crack propagation rate of the laminated structure according to claim 3, wherein,
the resultant force of the internal stresses and the resultant force of the bending moment and the shear stress of the upper sub-beam and the lower sub-beam are calculated as below:
based on the strain calculation formula of the upper sub-beam and the lower sub-beam:
$${ε}_{x}^{r} ( x , {z}^{r} ) = {u}_{0 , x}^{r} - {z}^{r} {φ}_{, x}^{r} \qquad ( 3 )$$
$${γ}_{x z}^{r} ( x , {z}^{r} ) = {w}_{, x}^{r} - {φ}^{r}$$
wherein
$${ε}_{x}^{r}$$
is a normal strain of the upper sub-beam and the lower sub-beam,
$${γ}_{xz}^{r}$$
is a shear strain of the upper sub-beam and the lower sub-beam, and in the variable subscript, x is derivation for corresponding variables;
the followings are obtained:
$${N}_{x}^{r} = b {\int }_{- \frac{{h}_{r}}{2}}^{\frac{{h}_{r}}{2}} {σ}_{x}^{r} {dz}^{r} = {A}_{11}^{r} {u}_{0 , x}^{r} - {B}_{11}^{r} {φ}_{, x}^{r} \qquad ( 4 )$$
$${M}_{x}^{r} = b {\int }_{- \frac{{h}_{r}}{2}}^{\frac{{h}_{r}}{2}} {σ}_{x}^{r} {z}^{r} {dz}^{r} = {B}_{11}^{r} {u}_{0 , x}^{r} - {D}_{11}^{r} {φ}_{, x}^{r}$$
$${V}_{xz}^{r} = b {\int }_{- \frac{{h}_{r}}{2}}^{\frac{{h}_{r}}{2}} {τ}_{xz}^{r} d {z}^{r} = {A}_{55}^{r} ( {w}_{, x}^{r} - {φ}^{r} )$$
wherein
$${N}_{x}^{r}$$
is the resultant force of the internal stresses of the upper sub-beam and the lower sub-beam,
$${M}_{x}^{r}$$
is the bending moment of the upper sub-beam and the lower sub-beam, and
$${V}_{x z}^{r}$$
is the resultant force of the shear stresses of the upper sub-beam and the lower sub-beam;
wherein b is a width of the laminated structure,
$${σ}_{x}^{r}$$
is a normal stress of the X axis direction,
$${τ}_{x z}^{r}$$
is a shear stress in an XZ plane,
$${A}_{11}^{r}$$
is a tensile stiffness of the upper sub-beam and the lower sub-beam,
$${B}_{11}^{r}$$
is a tensile-bending coupling stiffness of the upper sub-beam and the lower sub-beam,
$${D}_{11}^{r}$$
is a bending stiffness of the upper sub-beam and the lower sub-beam, and
$${A}_{55}^{r}$$
is a shear stiffness of the upper sub-beam and the lower sub-beam.

5. The estimation method of the fatigue crack propagation rate of the laminated structure according to claim 4, wherein,
the interface fracture toughness of the laminated structure is calculated as below:
$${ϕ}_{n} = \frac{3 P W}{2 ( a + Δ ) b} \qquad ( 5 )$$
wherein φₙ is an interface fracture toughness of the laminated structure, P is an applied load test value of the upper sub-beam, W is a displacement test value of the upper sub-beam, a is a theoretically-calculated crack length of the laminated structure, and Δ is a crack correction length.

6. The estimation method of the fatigue crack propagation rate of the laminated structure according to claim 5, wherein,
the tangential traction force and the normal traction force of the delamination crack interface are calculated as below:
$${T}_{t} ( D ) = \frac{2 {ϕ}_{n}}{{δ}_{0}} ( 1 - D ) \frac{Δ {u}_{t}}{{δ}_{0}} ( 1 + \frac{Δ {u}_{n}}{{δ}_{0}} ) exp ( - \frac{Δ {u}_{n}}{{δ}_{0}} - \frac{{( Δ {u}_{t} )}^{2}}{{( {δ}_{0} )}^{2}} ) \qquad ( 6 )$$
$${T}_{n} ( D ) = \frac{{ϕ}_{n}}{{δ}_{0}} ( 1 - D ) \frac{Δ {u}_{n}}{{δ}_{0}} exp ( - \frac{Δ {u}_{n}}{{δ}_{0}} - \frac{{( Δ {u}_{t} )}^{2}}{{( {δ}_{0} )}^{2}} )$$
wherein Tₜ is a tangential traction force, Tₙ is a normal traction force, δ₀ is a characteristic length of a cohesive zone, and D is a damage variable;
D=k∫max(\hat{D}_{c},\hat{D}ₘ)dt (7)
wherein k is a correction coefficient, \hat{D}_{c} is a cycle damage increment, \hat{D}_{c} is a monotonic damage increment, and t is time;
$${\hat{D}}_{c} = \frac{\vert Δ \hat{\overline{u}} \vert}{{δ}_{Σ}} [ \frac{\overline{T}}{{σ}_{m ax}} - \frac{{σ}_{F}}{{σ}_{m ax , 0}} ] H ( Δ \overline{u} - {δ}_{f} ) \qquad ( 8 )$$
$${\hat{D}}_{m} = \frac{\vert Δ \hat{\overline{u}} \vert}{{δ}_{Σ}} H ( Δ \overline{u} - {δ}_{0} ) \qquad ( 9 )$$
wherein Δ\hat{ū} is a cumulative displacement increment, δ_{Σ} is a total displacement amount required for the failure of the cohesive zone, T̅ is a total cohesive traction of the delamination crack interface, σ_{f} is a cohesive fatigue limit, σₘₐₓ is a normal cohesive strength after N load cycles, σ_{max,0} is an interfacial cohesive strength at the time of no damage, H is a Heaviside function, Δū is a cumulative displacement amount, and δ_{f} is a separation displacement corresponding to the cohesive fatigue limit;
$$H ( Δ \overline{u} - {δ}_{0} ) = \{ \begin{matrix}1 , & Δ \overline{u} - {δ}_{0} \geq 0 \\ 0 , & Δ \overline{u} - {δ}_{0} < 0\end{matrix} \qquad ( 10 )$$
wherein the cohesive fatigue limit σ_{f} is determined by a conditional yield stress: defining a parallel line of an elastic response stage of a curve of the normal traction force Tₙ−the normal displacement Δuₙ of the crack interface such that the parallel line is offset as per a designated plastic deformation amount d; the cohesive fatigue limit σ_{f} is jointly determined by dimensionless parameters δ_{f}/δ₀ and σ_{f}/σ_{max,0} at an intersection point of the offset parallel line and the curve of the normal traction force Tₙ−the normal displacement Δuₙ of the crack interface.

7. The estimation method of the fatigue crack propagation rate of the laminated structure according to claim 6, wherein,
the total strain energy of the laminated structure is calculated as below:
$$U = {\int }_{{V}^{t}} ( {σ}_{x}^{t} {ε}_{x}^{t} + {τ}_{xz}^{t} {γ}_{xz}^{t} ) {dV}^{t} + {\int }_{{V}^{b}} ( {σ}_{x}^{b} {ε}_{x}^{b} + {τ}_{xz}^{b} {γ}_{xz}^{b} ) {dV}^{b} + {\int }_{A} ( {T}_{t} Δ {u}_{t} + {T}_{n} Δ {u}_{n} ) dA \qquad ( 11 )$$
wherein U is the total strain energy of the laminated structure, V^{t} is a volume of the upper sub-beam, V^{b} is a volume of the lower sub-beam, A is an area between crack interface layers;
the potential energy of the external load applied to the laminated structure is expressed as V:
$$V = - {\int }_{0}^{L} {P}^{t} {w}^{t} {δ}_{D} ( x - {x}_{e} ) dx \qquad ( 12 )$$
wherein L is a length of the laminated structure, P^{t} is an applied load theoretical value of the upper sub-beam, δ_{D} is Dirac function, and xₑ is an X axis coordinate of a load application position;
$${δ}_{D} ( x - {x}_{e} ) = \{ \begin{matrix}1 , & x - {x}_{e} = 0 \\ 0 , & x - {x}_{e} \neq 0.\end{matrix} \qquad ( 13 )$$

8. The estimation method of the fatigue crack propagation rate of the laminated structure according to claim 7, wherein,
based on variational method, the equilibrium equation of the laminated structure is established:
δ(U−V)=0 (14)
wherein δ is a variational symbol;
the following set of governing equations is obtained based on the equilibrium equation of the structure:
$${N}_{x , x}^{t} = - b {T}_{t} \qquad ( 15 )$$
$${V}_{xz , x}^{t} = - b {T}_{n} \qquad ( 16 )$$
$${M}_{x , x}^{t} = {V}_{xz}^{t} - \frac{b {h}_{t}}{2} {T}_{t} \qquad ( 17 )$$
$${N}_{x , x}^{b} = b {T}_{t} \qquad ( 18 )$$
$${V}_{xz , x}^{b} = b {T}_{n} \qquad ( 19 )$$
$${M}_{x , x}^{b} = {V}_{xz}^{b} - \frac{{bh}_{b}}{2} {T}_{t} \qquad ( 20 )$$
$${u}_{0 , x}^{t} = \frac{{D}_{11}^{t} {N}_{x}^{t} - {B}_{11}^{t} {M}_{x}^{t}}{{A}_{11}^{t} {D}_{11}^{t} - {( {B}_{11}^{t} )}^{2}} \qquad ( 21 )$$
$${w}_{0 , x}^{t} = {φ}^{t} + \frac{{V}_{xz}^{t}}{{A}_{55}^{t}} \qquad ( 22 )$$
$${φ}_{, x}^{t} = \frac{{B}_{11}^{t} {N}_{x}^{t} - {A}_{11}^{t} {M}_{x}^{t}}{{A}_{11}^{t} {D}_{11}^{t} - {( {B}_{11}^{t} )}^{2}} \qquad ( 23 )$$
$${u}_{0 , x}^{b} = \frac{{D}_{11}^{b} {N}_{x}^{b} - {B}_{11}^{b} {M}_{x}^{b}}{{A}_{11}^{b} {D}_{11}^{b} - {( {B}_{11}^{b} )}^{2}} \qquad ( 24 )$$
$${w}_{0 , x}^{b} = {φ}^{b} + \frac{{V}_{x z}^{b}}{{A}_{55}^{b}} \qquad ( 25 )$$
$${φ}_{, x}^{b} = \frac{{B}_{11}^{b} {N}_{x}^{b} - {A}_{11}^{b} {M}_{x}^{b}}{{A}_{11}^{b} {D}_{11}^{b} - {( {B}_{11}^{b} )}^{2}} . \qquad ( 26 )$$

9. The estimation method of the fatigue crack propagation rate of the laminated structure according to claim 8, wherein,
the set of governing equations is solved to obtain the crack length a and the cycle number N of the laminated structure and the applied-load theoretical value P^{t} of the upper sub-beam and the displacement theoretical value w^{t} of the upper sub-beam under the fatigue load of the laminated structure;
the cyclic energy release rate is expressed as ΔG:
$$Δ G = \frac{{( {P}_{m ax}^{t} )}^{2} - {( {P}_{m i n}^{t} )}^{2}}{2 b} \frac{dC}{da} \qquad ( 27 )$$
wherein
$${P}_{m ax}^{t}$$
is a maximum value of the applied load theoretical value of the upper sub-beam,
$${P}_{m i n}^{t}$$
is a minimum value of the applied load theoretical value of the upper sub-beam, and C is a compliance of the laminated structure; C=w^{t}/P^{t};
fitting is performed on the theoretically-calculated crack length a and the cycle number N of the laminated structure by using power function to obtain a functional relation and then differential operation is performed on the functional relation to obtain the crack propagation rate da/dN.

10. The estimation method of the fatigue crack propagation rate of the laminated structure according to claim 9, wherein,
the crack propagation rate da/dN and the cyclic energy release rate ΔG are mapped on a double-logarithmic coordinate system to obtain a curve of the fatigue crack propagation rate of the laminated structure; then based on Paris law, fitting is performed on the curve of the fatigue crack propagation rate to obtain a fitted curve; based on the fitted curve, an actual fatigue crack propagation rate is determined:
$$\frac{d a}{d N} = {B ( Δ G )}^{m} \qquad ( 28 )$$
wherein B is a material-related constant and m is an exponential constant of the Paris law.
